(19) United States
(12) Patent Application Publication
(10) Pub. No.: US 2021/0038982 A1
(43) Pub. Date: Feb. 11, 2021

(54) SYSTEM, COMPUTING DEVICE, AND METHOD FOR MAPPING AN ACTIVITY OF A PLAYER IN A NON-VIRTUAL ENVIRONMENT INTO A VIRTUAL ENVIRONMENT

(71) Applicant: INTELLISPORTS INC., Montréal (CA)

(72) Inventors: Jonathan GUILLEMETTE, Montréal (CA); Benjamin F. MATTES, Montréal (CA)

(21) Appl. No.: 16/965,570

(22) PCT Filed: Jan. 29, 2019
(86) PCT No.: PCT/CA2019/050105

§ 371 Date: Jul. 28, 2020

Related U.S. Application Data
(60) Provisional application No. 62/623,178, filed on Jan. 29, 2018.

(51) Int. Cl. A63F 13/428; A63F 13/211; A63F 13/212; A63F 13/65; A63F 13/798

(52) U.S. Cl. CPC A63F 13/428; A63F 13/211; A63F 2300/8082; A63F 13/65; A63F 13/798; A63F 13/212

(57) ABSTRACT

A system and method for mapping a play activity performed by a player in a non-virtual environment into a virtual gaming environment are provided. At least one data collector is configured to collect, at least during the play activity, data indicative of a performance of the player. A computing device obtains, from the at least one data collector, the data indicative of the performance of the player and updates, based on the obtained data, one or more characteristics associated with a virtual representation of the player within a virtual environment of a virtual game.

### CROSS REFERENCE TO RELATED APPLICATIONS

This patent application claims priority of U.S. provisional Application Ser. No. 62/623,178, filed on Jan. 29, 2018, the entire contents of which are hereby incorporated by reference.

### TECHNICAL FIELD

The application relates generally to objects of play and, more particularly, to mapping a play activity performed in a non-virtual environment into a virtual environment.

### BACKGROUND

Intelligent gaming objects, such as balls, pucks, discs, and sticks, as well as data collectors provided on the body of a player can be used to collect information about the movement of the gaming object or the player. This information can be transmitted (e.g., from the gaming object) and analysed to obtain information about the player's skills. Still, challenges remain, such as motivating players to use the objects on a consistent and ongoing basis. The information collected and transmitted is also often insufficient to be used to adequately assess a player's skills. In addition, the correctional guidance that can be provided to the player is limited. There is therefore room for improvement.

### SUMMARY

In accordance with a broad aspect, there is provided a system for mapping a play activity performed by a player in a non-virtual environment into a virtual gaming environment. The system comprises at least one data collector configured to collect, at least during the play activity, data indicative of a performance of the player and a computing device comprising at least one processing unit and at least one non-transitory computer-readable memory having stored thereon a virtual game and a virtual representation of the player within a virtual environment of the virtual game, the virtual representation of the player having one or more characteristics associated therewith. The memory also has stored thereon instructions executable by the at least one processing unit for obtaining, from the at least one data collector, the data indicative of the performance of the player, and updating, based on the data indicative of the performance of the player, the one or more characteristics associated with the virtual representation of the player.

In some embodiments, the at least one data collector is at least one play object manipulable by the player during the play activity.

In some embodiments, the at least one play object is a connected sports object.

In some embodiments, the at least one data collector is at least one motion sensing device located on a body of the player.

In some embodiments, the at least one data collector is at least one motion sensing device provided in a portable communication device of the player.

In some embodiments, the at least one play object comprises a data collecting unit configured to collect the data indicative of the performance of the player and to output the data to the computing device.

In some embodiments, the data collecting unit comprises at least one accelerometer unit configured for measuring acceleration values of the at least one play object along at least one translational degree of freedom, at least one gyroscope unit configured for measuring rotation values of the at least one play object about at least one rotational degree of freedom, and a processor in communication with the at least one accelerometer unit and with the at least one gyroscope unit, the processor configured for obtaining the acceleration values from the at least one accelerometer unit, obtaining the rotation values from the at least one gyroscope unit, and outputting the acceleration values and the rotation values to the computing device at discrete intervals.

In some embodiments, the at least one processing unit is configured for receiving the acceleration values and the rotation values and for determining therefrom the data indicative of the performance of the player.

In some embodiments, the data collecting unit further comprises a power source configured for supplying electrical power to the at least one accelerometer unit, the at least one gyroscope unit, and the processor.

In some embodiments, the data collecting unit further comprises a transmitting unit configured for wirelessly transmitting the acceleration values and the rotation values to the computing device.

In some embodiments, the instructions are executable by the at least one processing unit for updating the one or more characteristics comprising increasing or decreasing a rank associated with the virtual representation of the player in the virtual game.

In some embodiments, the instructions are executable by the at least one processing unit for updating the one or more characteristics comprising increasing or decreasing a skill level associated with the virtual representation of the player in the virtual game.

In some embodiments, the instructions are executable by the at least one processing unit for updating the one or more characteristics based on a duration of time that the player spends performing the play activity.

In some embodiments, the instructions are executable by the at least one processing unit for updating the one or more characteristics comprising increasing a duration of play time associated with the virtual representation of the player in the virtual game.

In some embodiments, the system further comprises an audio output device wearable by the player, the instructions executable by the at least one processing unit for outputting in substantially real-time, via the audio output device, audio status information about any update to the virtual representation of the player.

In some embodiments, the computing device further comprises a display unit configured to render thereon the data indicative of the performance of the player.

In accordance with another broad aspect, there is provided a computer-implemented method for mapping a play activity performed by a player in a non-virtual environment into a virtual gaming environment. The method comprises, at a computing device, forming a virtual representation of the player within a virtual environment of a virtual game, the virtual representation of the player having one or more characteristics associated therewith, obtaining, from at least one data collector, data indicative of a performance of the player, and updating the one or more characteristics associated with the virtual representation of the player based on the data as obtained.

In some embodiments, obtaining the data indicative of the performance of the player comprises obtaining, from at least one accelerometer unit, acceleration values of the at least one data collector along at least one translational degree of freedom, obtaining, from at least one gyroscope unit, rotation values of the at least one data collector about at least one rotational degree of freedom, and determining the data indicative of the performance of the player from the acceleration values and the rotation values.

In some embodiments, updating the one or more characteristics comprises increasing or decreasing a rank associated with the virtual representation of the player in the virtual game.

In some embodiments, updating the one or more characteristics comprises increasing or decreasing a skill level associated with the virtual representation of the player in the virtual game.

In some embodiments, the one or more characteristics are updated based on a duration of time that the player spends performing the play activity.

In some embodiments, updating the one or more characteristics comprises increasing a duration of play time associated with the virtual representation of the player in the virtual game.

In some embodiments, the method further comprises outputting in substantially real-time, via an audio output device wearable by the player, audio status information about any update to the virtual representation of the player.

In some embodiments, the method further comprises rendering the data indicative of the performance of the player on a display unit.

In accordance with another broad aspect, there is provided a computing device comprising at least one processing unit and at least one non-transitory computer-readable memory having stored thereon a virtual game and a virtual representation of the player within a virtual environment of the virtual game, the virtual representation of the player having one or more characteristics associated therewith. The memory also has stored thereon instructions executable by the at least one processing unit for obtaining, from the at least one data collector, data indicative of a performance of a player during a play activity, and updating the one or more characteristics associated with the virtual representation of the player based on the data as obtained.

In some embodiments, the instructions are executable by the at least one processing unit for obtaining the data indicative of the performance of the player comprising obtaining, from at least one accelerometer unit, acceleration values of the at least one play object along at least one translational degree of freedom, obtaining, from at least one gyroscope unit, rotation values of the at least one play object about at least one rotational degree of freedom, and determining the data indicative of the performance of the player from the acceleration values and the rotation values.

In some embodiments, the instructions are executable by the at least one processing unit for updating the one or more characteristics comprising increasing or decreasing a rank associated with the virtual representation of the player in the virtual game.

In some embodiments, the instructions are executable by the at least one processing unit for updating the one or more characteristics comprising increasing or decreasing a skill level associated with the virtual representation of the player in the virtual game.

In some embodiments, the instructions are executable by the at least one processing unit for updating the one or more characteristics based on a duration of time that the player spends performing the play activity.

In some embodiments, the instructions are executable by the at least one processing unit for updating the one or more characteristics comprising increasing a duration of play time associated with the virtual representation of the player in the virtual game.

In some embodiments, the instructions are further executable by the at least one processing unit for forming a virtual team in the virtual game, members of the virtual team comprising the virtual representation of the player and at least one additional virtual representation of at least one other player.

In some embodiments, the instructions are further executable by the at least one processing unit for creating, in the virtual game, a virtual match between the virtual team as formed and one additional virtual team.

In some embodiments, the instructions are further executable by the at least one processing unit for outputting in substantially real-time, via an audio output device wearable by the player, audio status information about any update to the virtual representation of the player.

In some embodiments, the instructions are further executable by the at least one processing unit for rendering the data indicative of the performance of the player on a display unit.

In accordance with another broad aspect, there is provided a non-transitory computer-readable medium having stored thereon a virtual game and a virtual representation of a player within a virtual environment of the virtual game, the virtual representation of the player having one or more characteristics associated therewith. The computer-readable medium also has stored thereon program code executable by at least one processor for obtaining, from at least one data collector, data indicative of a performance of a player during a play activity, and updating the one or more characteristics associated with the virtual representation of the player based on the data as obtained.

### DESCRIPTION OF THE DRAWINGS

Reference is now made to the accompanying figures in which:

FIG. 1 is a schematic view of a system for tracking a player performing a play activity in a non-virtual environment and mapping the play activity into a virtual environment, according to an embodiment of the present disclosure;

FIG. 2A is a perspective view of a play object of the system of FIG. 1, the play object having a data-collecting unit;

FIG. 2B is schematic view of the data-collecting unit of FIG. 2A;

FIG. 3 is a schematic view of a mobile computing device of the system of FIG. 1;

FIGS. 4A to 6C are schematic views of pages of a virtual game executed on a mobile computing device of the system of FIG. 1; and

FIG. 7 is a flowchart of a method for tracking a player performing a play activity in a non-virtual environment and mapping the play activity into a virtual environment.

### DETAILED DESCRIPTION

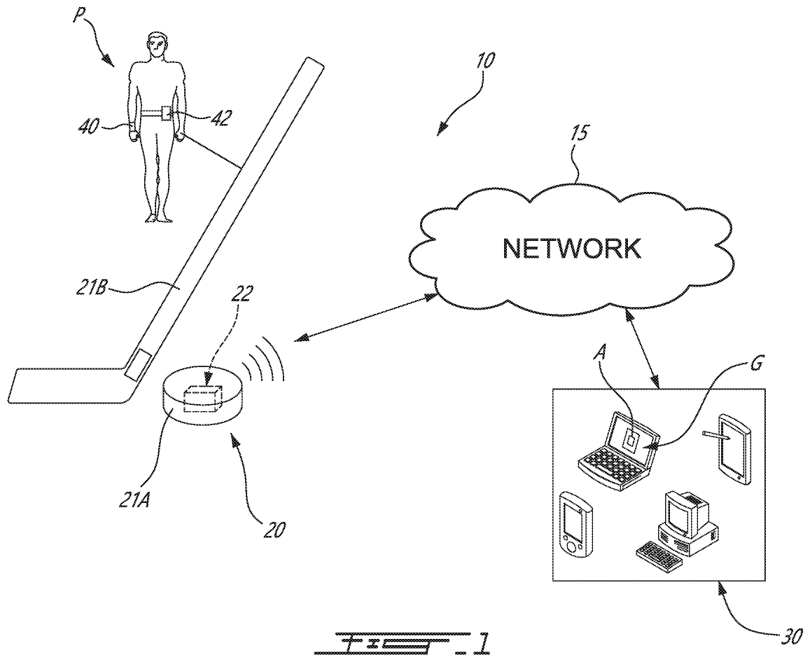

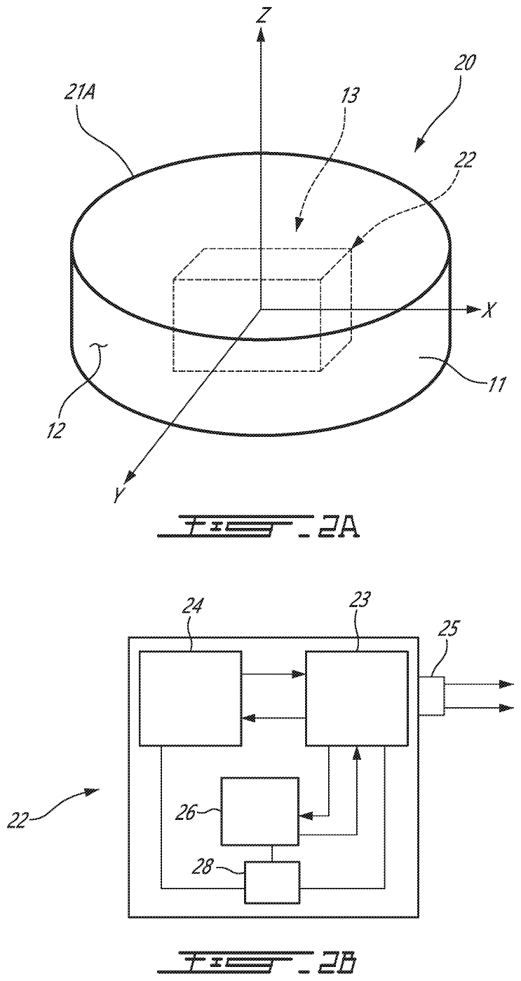

FIG. 1 illustrates a system 10 for tracking a player P performing one or more movements (also referred to herein as “user-generated motion”) in a non-virtual environment and mapping the user-generated motion into a virtual environment, in accordance with one embodiment. The user-generated motion may be performed as part of a given physical activity (also referred to herein as a “play activity”), which may be any suitable activity including, but not limited to a sport activity. When performing the play activity, the player P generates data that is captured by the system 10. The data generated by the player P (also referred to herein as “motion data”) and captured by the system 10 is used by the system 10 to provide enjoyment and/or a benefit to the player P, in the form of a virtual game G executed on a computational medium. More particularly, the system 10 and its components use the data generated by the player P during the real-world activity to update a virtual representation of the player P in the virtual game G executed on the computational medium. The updated virtual version or representation of the player P, referred to herein as an avatar A of the player P, performs activities within the virtual game G. The system 10 therefore helps to map the physical activity of the player P in the real, non-virtual world onto the avatar A within the virtual environment of the virtual game G. In so doing, the system 10 helps the player P to link or map a real person, environment, or activity to virtual ones.

The system 10 has at least the following two components: a device for generating motion data from the play activity of the player P, and a device (referred to herein as a “data collector”) for capturing the motion data generated by the player P and updating the avatar A. In one embodiment, the data collector is a play object 20 which is manipulated by the player P in the real, non-virtual environment to generate data. In another embodiment, a plurality of independent data collectors (e.g., wearable sensing devices) as in 40, 42 are provided at various locations on the player's body (or clothing) or in a personal electronic device (not shown) of the player P and configured to collect the motion data generated during the play activity. The personal electronic device may comprise any portable or handheld communication device, such as a mobile phone, a smartphone, a personal digital assistant (PDA), or the like, adapted to communicate over a network. A computing device 30 which communicates with the play object 20 (and/or data collectors as in 40, 42, and/or the player's personal electronic device), contains the virtual game G and avatar A, and updates the avatar A based on the data provided by the play object 20 (and/or data collectors as in 40, 42, and/or the player's personal electronic device).

Still referring to FIG. 1, the play object 20 has a data-collecting unit 22 which generates data, and transmits data to the computing device 30 when the play object 20 is manipulated by the player P during the play activity. FIG. 1 shows a single play object 20 being manipulated by the player P. In an alternate embodiment, the system 10 includes multiple play objects 20 manipulated by the player P during the play activity. The expression “play activity” refers to any physical activity performed by the player P in a real or non-virtual environment. In the embodiment where one or more play objects 20 are manipulated by the player P during the play activity, the expression “play activity” refers to any movement or action of the play object 20 caused by the player P. Some non-limiting examples of play activities performed with, on, or against the play object 20 include, but are not limited to, running, shooting, sliding, moving, punching, throwing, jumping, handling, swinging, skating, dribbling, and passing. For example, in the depicted embodiment, the play object 20 is a hockey puck 21A which is manipulated by the player P using a hockey stick 21B. Some non-limiting examples of play activities that the player P may perform with, on, or against the hockey puck 21A with the hockey stick 21B include shooting, skating, passing, and stick handling. Other examples of play activities include, but are not limited to, lunges, push-ups, squats, or any other user-generated motion, which may be performed by the player P without involving a play object 20. In this case, the player's motion and general fitness level could also be mapped onto the avatar A.

In the depicted embodiment, the data-collecting unit 22 is operable during manipulation of the play object 20 by the player P to wirelessly transmit data indicative of player performance to the computing device 30. In this embodiment, the data may be transmitted to the computing device 30 in substantially real-time. In an alternate embodiment, the data-collecting unit 22 stores the data indicative of player performance for a given period of time (e.g., a duration of the play activity) so that it may be downloaded from the play object 20 and provided to the computing device 30 (e.g., at the end of the given period of time). The data indicative of player performance and player use is information related to the skill and/or usage of the player P when manipulating the play object 20. Some examples of data indicative of player performance are described below. In the depicted embodiment, and as described in greater detail below, the data-collecting unit 22 transmits raw, unprocessed information generated by the data-collecting unit 22 to the computing device 30, which then transforms the unprocessed information into data indicative of player performance. In an alternate embodiment, the data-collecting unit 22 has computational capacity to generate unprocessed data, analyse the unprocessed data, and output the data indicative of player performance to the computing device 30. It will therefore be appreciated that the expression “data indicative of player performance” includes information related to the skill or abilities of the player P when manipulating the play object 20, and also includes raw or unprocessed data that can be processed into information related to the skill or abilities of the player P when manipulating the play object 20. In at least the depicted embodiment, the play object 20 is a connected sports object, or “CSO”. It should however be understood that the player P may manipulate their personal electronic device (e.g., a mobile phone) while performing the play activity. Therefore, the player's personal electronic device (e.g., mobile phone) may be considered a play object as in 20.

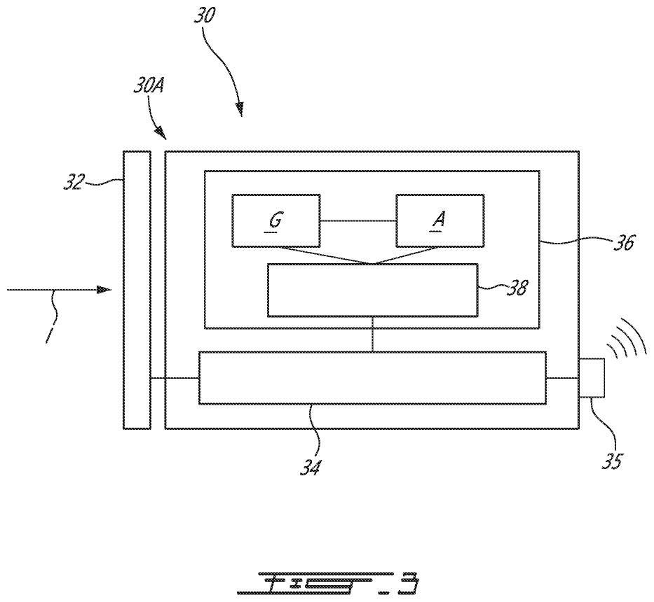

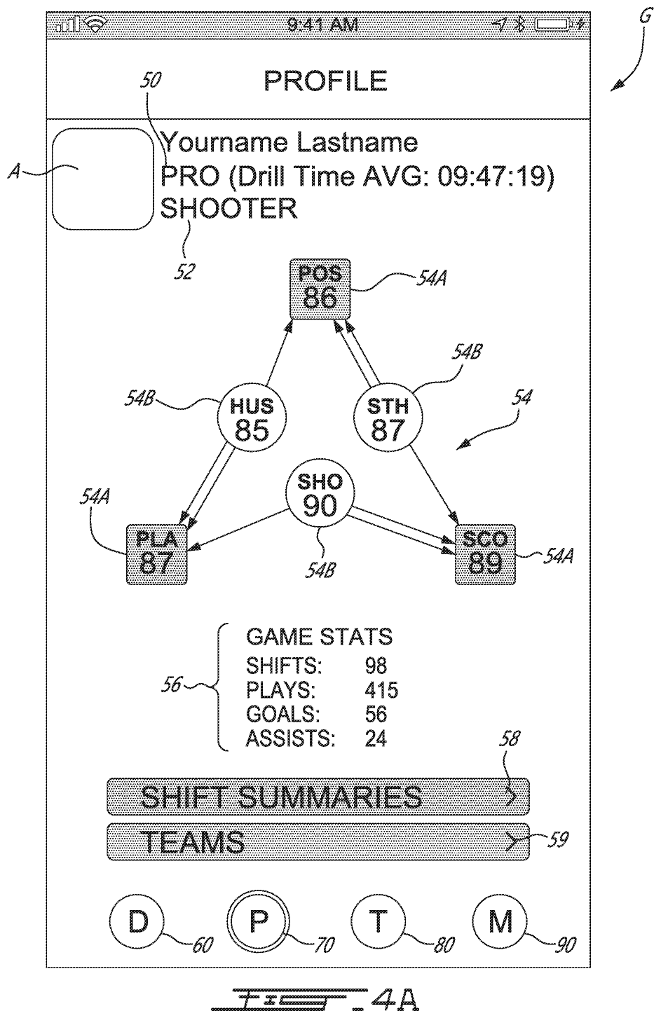

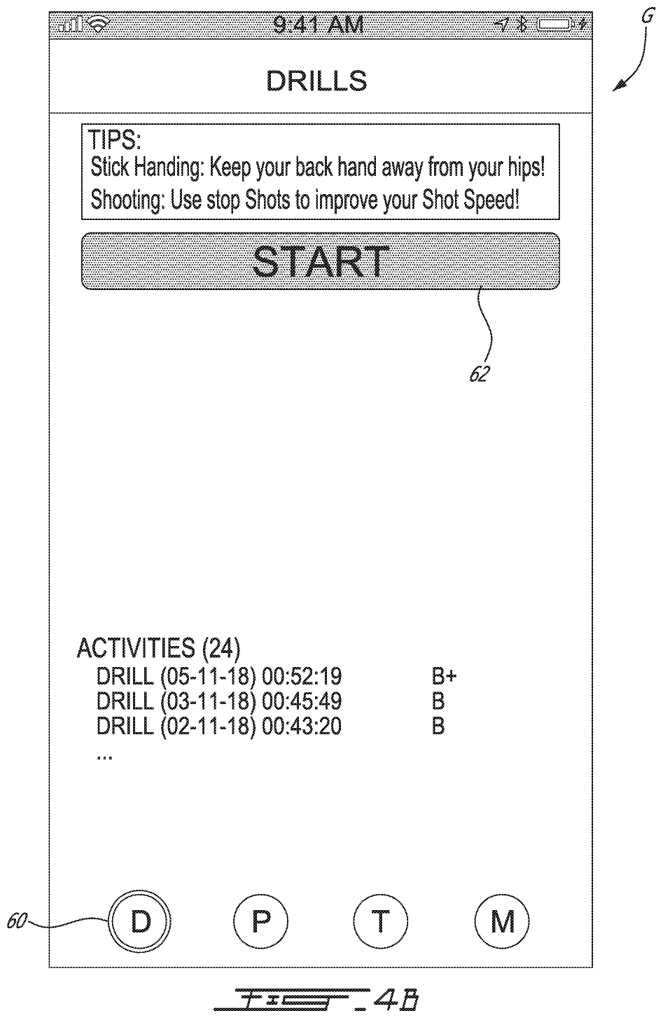

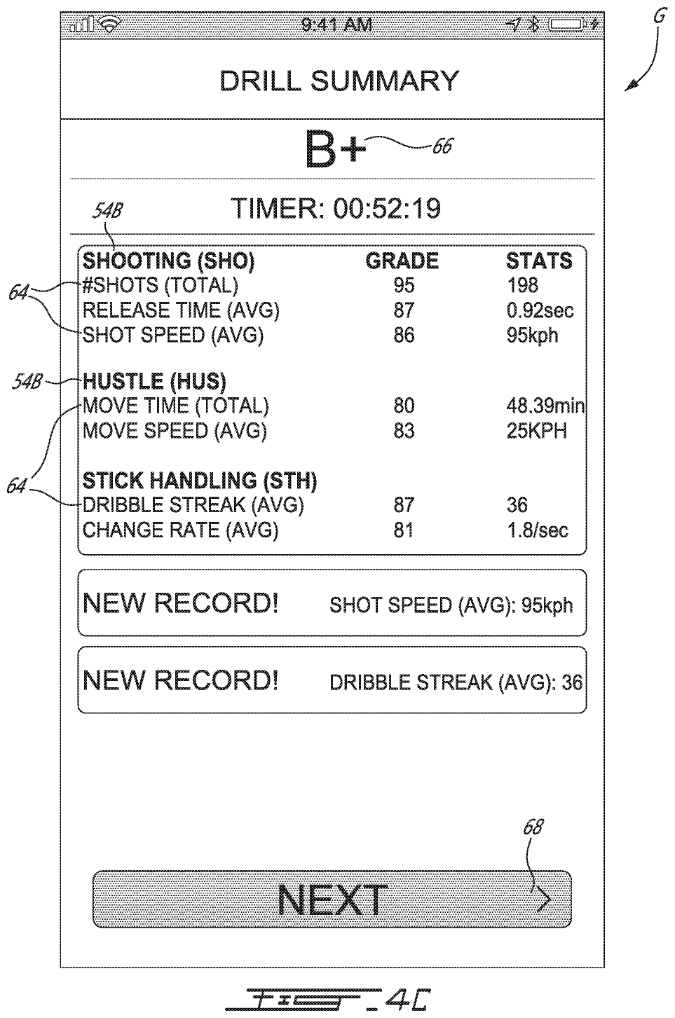

FIGS. 2A and 2B show the play object 20 for collecting and transmitting the data indicative of player performance. The data collected and transmitted can be analysed to provide information about the skills of the player P using the play object 20. The play object 20 may therefore serve as a diagnostic or analytic tool for evaluating player performance. The play object 20 is described in detail in PCT patent application having application number PCT/CA2016/051137 and publication number WO 2017/054082, entitled “DATA-COLLECTING PLAY OBJECT, SYSTEM AND METHOD” and filed Sep. 30, 2016, the entire contents of which are hereby incorporated by reference.

The play object 20 can be any object or device used during sports or activities. The play object 20 is manipulated during use, either directly or indirectly, by the player P such that it undergoes movement. Some non-limiting examples of play objects 20 included in the scope of the present disclosure include a ball (baseball, softball, golf, lacrosse, cricket, bowling, football, soccer ball, boxing gloves, basketball, etc.), a disc (i.e. such as a Frisbee™), a puck, a baseball bat, a hockey stick, a curling rock, and a boxing glove. Some of the play objects 20 in the preceding list of examples have solid inner cores, and do not have hollow interiors. Other play objects 20 in the preceding list of examples have hollow interiors, typically filled with air. In the embodiment of FIG. 2A, the play object 20 is the hockey puck 21A. Reference herein to hockey pucks or skills associated with hockey does not limit the disclosed play object 20 to being only a hockey puck, or to being used only in the sport of hockey. In an alternate embodiment, the play object 20 is wearable by the player P.

The play object 20 has a body 11, which forms the corpus of the play object 20 and provides structure thereto. An outer surface 12 of the body 11 is typically manipulated by the player P to displace the play object 20, either directly by the player's hand or indirectly via an intermediate object such as a stick 21B or a bat. In the embodiment shown where the play object 20 is a hockey puck 21A, the outer surface 12 is cylindrical with flat upper and lower surfaces. In the depicted embodiment, the body 11 also has an interior 13 which includes a solid inner core.

The play object 20 has six degrees of freedom and is manipulated to be moved therein. More particularly, the play object 20 has three translational degrees of freedom in which it is displaced, and three rotational degrees of freedom about which it rotates. These degrees of freedom are more easily appreciated by referring to the play object's 20 own coordinate system, defined by three orthogonal axes of motion, namely an X axis, a Y axis, and a Z axis. The three translational degrees of freedom are displacement movements of the play object along the X, Y, and Z axes. In the depicted embodiment, the X and Y axes define movement along a horizontal plane, and the Z axis is vertically oriented and defines movement in a vertical direction. The three rotational degrees of freedom are rotational movements about the X, Y, and Z axes.

The data-collecting unit 22 is disposed in the interior 13 of the body 11 of the play object 20. In the depicted embodiment, the data-collecting unit 22 is part of the solid inner core of the body 11. The data-collecting unit 22 collects data related to the movement of the play object 20, and transmits the data to a separate and remote device or system, such as the computing device 30, so that it can be analysed to provide information on player performance. This movement data can vary, and is data related to the displacement of the play object 20 about itself, through space, and in time. It will be appreciated that the data-collecting unit 22 may also be operational when the play object 20 is stationary and not being manipulated.

The location of the data-collecting unit 22 within the play object 20 can vary, depending on the type of play object 20 being used and the nature of movement data being collected. For example, in the embodiment of FIGS. 2A and 2B where the play object 20 is a hockey puck 21A, the data-collecting unit 22 is fixedly secured in place within the interior 13 of the hockey puck 21A itself, as part of its inner core. For other types of play objects 20, the data-collecting unit 22 can be rigidly disposed within the play object 20 using any suitable technique.

Referring to FIG. 2B, the data-collecting unit 22 measures the movement of the play object 20 with one or more accelerometer units 24, and one or more gyroscope units 26. A processor 23 communicates with the accelerometer and gyroscope units 24, 26 and transmits their measured values (referred to herein as acceleration values and rotation values, respectively) away from the play object 20 to the computing device 30. A power source 28 provides electrical power to each of the accelerometer unit 24, the gyroscope unit 26, and the processor 23. These components of the data-collecting unit 22 are now discussed in greater detail.

The accelerometer unit 24 measures the movement of the play object 20 by generating its acceleration values along one or more of the three translational degrees of freedom. “Acceleration values” are understood herein to include acceleration vectors, as well as time derivatives/integrals of these values such as speed and displacement. The acceleration values therefore have information on the direction of acceleration along any one of the X, Y, and Z axes, as well as the magnitude of acceleration. The accelerometer unit 24 outputs the acceleration values in units of distance per unit of time squared (e.g. in/s², cm/s², ft/s², m/s², etc.). For example, it may be possible to determine the velocity and speed of the play object 20, along any one of the X, Y, and Z axes, from the measured acceleration values. It is similarly possible to determine the distance travelled by the play object 20 along any one of the axes from the measured acceleration values. If the starting point of the play object 20 is known or provided by the player P, the distance can be used to determine the displacement of the play object 20 within another coordinate system at any given moment in time. It can thus be appreciated that the accelerometer unit 24 can be any device capable of such functionality, and typically includes an accelerometer and an associated memory or processor.

Still referring to FIG. 2B, the accelerometer unit 24 samples or collects data constantly, at discrete time intervals. The accelerometer unit 24 could measure the acceleration values at a relative high frequency, depending on the activity being measured. This sampling frequency can be in the range of 500 Hz to 2 kHz, for example, although other sampling frequencies are also within the scope of the present disclosure. The higher the sampling frequency, the more accurate the subsequent measurements will be, and hence the better the granularity of the data generated. The nature of the accelerometer unit 24 can also vary, depending on the type of play object 20 being used and the data being obtained. For example, in the embodiment where the play object 20 is a hockey puck 21A, the accelerometer unit 24 is a “high g” accelerometer unit 24, meaning that it is capable of measuring higher accelerations values in the order of hundreds of “g”. It will be appreciated that such a high g accelerometer unit 24 is capable of capturing lower acceleration values as well. As explained in greater detail below, this high g accelerometer unit 24 helps to capture movements of the play object 20 where it undergoes large accelerations, such as when taking a shot with the hockey puck 21A. In an alternate embodiment the accelerometer unit 24 is a “low g” accelerometer unit 24, meaning that it is capable of measuring lower accelerations values in the order of tens of “g”. This low g accelerometer unit 24 helps to capture movements of the play object 20 where it undergoes relative low accelerations, such as during stick handling of a hockey puck 21A or when the player P skates with the hockey puck 21A. It will be appreciated that other “g” values are within the scope of the present disclosure, and that the accelerometer unit 24 may have multiple accelerometer units 24, of both the high or low “g” types. Indeed, using both a high and low g accelerometer unit 24 enables every relevant movement of the play object 20 to be captured by the data-collecting unit 22 within a wide range of acceleration values.

Still referring to FIG. 2B, the gyroscope unit 26 measures the movement of the play object 20 by producing its rotation values about one or more of the three rotational degrees of freedom. “Rotation values” are understood herein to include measurements of the rotation or “spin” of the play object 20, as well as time derivatives/integrals of these values. The rotation values include information on the direction of rotation about any one of the X, Y, and Z axes, as well as the magnitude of rotation. For example, it is possible to determine the angular velocity or speed, as well as the RPM of the play object 20, about any one of the X, Y, and Z axes from the rotation values. The gyroscope unit 26 outputs the rotation values in units of angular displacement per unit of time (e.g. deg/s, rad/s, etc.).

Knowing the rotational speed may provide information about the stability of the play object 20 as it travels. For example, in the embodiment where the play object 20 is a hockey puck 21A, knowing the RPM of the puck in the Z axis, as well as variances in the RPM, is indicative of the stability or “straightness” of the hockey puck 21A as it travels through the air. The gyroscopic effect teaches that the hockey puck 21A rotating at a higher speed will be more stable in its plane of rotation than a hockey puck 21A rotating at a lower speed in the same plane because a small deviation applied to the rotating hockey puck 21A will be more quickly corrected in the faster rotating hockey puck 21A. It is also possible to determine the angular acceleration of the play object 20, which may be important to know for some play activities (e.g. rotation of a baseball), from the rotation values. The gyroscope unit 26 can be any device capable of such functionality, and typically includes a gyroscope and an associated memory or processor.

Still referring to FIG. 2B, the gyroscope unit 26 samples or collects data constantly, at discrete time intervals. The gyroscope unit 26 can measure the rotation values at a relative high frequency, an example of which is the range of about 500 Hz to about 1 kHz. This helps to ensure a high granularity of rotation values generated by the gyroscope unit 26 and transmitted by the play object 20. The nature of the gyroscope unit 26 can also vary, depending on the type of play object 20 being used and the data being obtained. A lower capacity gyroscope unit 26, such as one that can measure rotations in the order of hundreds of deg/s, may be useful for those play objects 20 for which it is not required or beneficial to measure high speeds of rotations. A higher capacity gyroscope unit 26, such as in the range of thousands of deg/s, may be useful for those play objects 20 for which it is beneficial to measure high speeds of rotations, such as during the rotation of a hockey puck 21A about the Z axis. An even higher capacity gyroscope unit 26, such as in the range of tens of thousands of deg/s, may be useful for those play objects 20 for which it is beneficial to capture very high speeds of rotation, such as those of a baseball thrown by an elite-level pitcher. It will therefore be appreciated that other “deg/s” values are within the scope of the present disclosure, and that the data-collecting unit 22 may have more than one gyroscope unit 26, of the both the high and low capacity types.

Both the accelerometer and gyroscope units 24, 26 may collect movement data along one or more of the X, Y, and Z axes. For example, and as discussed in greater detail below, for some types of player drills, it may be suitable to deactivate data collection along/about one or more of the axes. This can involve instructing the accelerometer or gyroscope units 24,26 to not generate data along/about the axis in question. This can also involve having the processor 23 ignore the data collected related to the axis in question, or to not transmit the data from this axis. Disregarding data from one or more axes may reduce data transmission and analysis delays. In other situations, it may be desired to collect data from all three axes, in which case the accelerometer and gyroscope units 24,26 can be referred to as “triple axis” accelerometer and gyroscope units 24, 26.

The processor 23 communicates with the accelerometer unit 24 and with the gyroscope unit 26 and obtains from them respectively the acceleration values and the rotation values. The processor 23 then transmits the acceleration and rotation values at discrete time intervals to a system or device which analyses this data, such as the computing device 30. The processor 23 in most embodiments, but not necessarily all, does not perform analysis itself of the raw, unprocessed acceleration and rotation values. In such an embodiment, where the processor 23 operates primarily to transmit the acceleration and rotation values, the processor 23 helps to lower the energy consumption of the data-collecting unit 22. As such, the processor 23 may be integral with the accelerometer unit 24 and with the gyroscope unit 26. The processor 23 may therefore be any device that can collect and transmit data. Some non-limiting examples of the processor 23 include a microcontroller, a central processing unit (CPU), a front-end processor, a microprocessor, a graphics processing unit (GPU/VPU), a physics processing unit (PPU), a digital signal processor, and a network processor. The processor 23 may also be part of a flexible PCB, which would allow the data-collecting unit 22 to match a curvature of a play object 20, such as the curvature of a baseball.

Still referring to FIG. 2B, the processor 23 transmits the acceleration and rotation values wirelessly. The transmission of the values is generally performed with a transmitting unit 25, such as an antenna or transceiver, to a remote device or network. In some embodiments, the transmitting unit 25 is a Bluetooth™ transmitter. The transmission frequency can vary from about 30 Hz to about 140 Hz, for example.

Although reference is made herein to a play object 20 being used to collect and transmit data indicative of player performance, it should be understood that the player P need not perform the play activity using the play object 20 and that the systems and methods described herein are not limited to the use of a play object as in 20. The motion data may indeed be collected from various locations on the user's body and/or the play object 20, using any suitable number of data collectors. For example, as shown in FIG. 1, a plurality of independent data collectors 40, 42 (e.g., wearable sensing devices) may be provided at various locations on the player's body (or clothing) to collect the motion data generated during the play activity. For example, a first data collector 40 may be attached to the player's wrist to collect data indicative of the player's limb (e.g. arm) motion. A second data collector 42 may be attached to the player's hip to collect data indicative of motion of the player's core. The data collectors 40, 42 may also be provided in the player's personal electronic device, which can be secured to the player's body via suitable attachment means (or handheld by the player P).

The data collectors 40, 42 can be used to measure the acceleration and rotation values and provide these values to the computing device 30. The data collectors 40, 42 may indeed comprise any suitable motion sensing devices configured to measure movement and output corresponding signal(s). For example, the data collectors 40, 42 include, but are not limited to, accelerators configured to produce acceleration values and gyroscopes configured to produce rotation values. As such, although reference is made herein to the data-collecting unit 22 of the play object 20 being configured to communicate acceleration and rotation values to the computing device 30, it should be understood that the acceleration and rotation values may be provided by the data collectors 40, 42.

The acceleration and rotation values obtained from the data collectors 40, 42 can then be used by the computing device 30 to determine player performance. For instance, when the play activity is a push-up, the computing device 30 may take the double integral of the acceleration obtained from the data collectors 40, 42 (i.e. accelerometers) in order to determine a distance traveled by the personal electronic device (and accordingly by the player P) during the push-up. The distance may then be used to grade the push-up. Indeed, based on the computed distance, it becomes possible to determine whether the player P has performed a deep or shallow push-up. In another example, if the player P is doing a squat, the computing device 30 may measure the oscillation of the acceleration signal obtained from the data collectors 40, 42 (i.e. accelerometers) in order to determine the player's performance while doing the squat. In yet another example, if the motion being performed by the player P is a baseball pitch, the computing device 30 may calculate the integral of the rotation values obtained from the data collectors 40, 42 (i.e. gyroscopes) in order to obtain the angle swept by the player's hip movement. The angle may then be used to grate the pitch. Indeed, it may be possible to determine how much angular velocity the player P had and for what period of time and to accordingly determine if the player P threw the pitch with as much rhythm as usual (i.e. compared to previous pitches). Other embodiments may apply. It should therefore be understood that reference herein to using a play object as in 20 to collect and transmit data indicative of player performance does not limit the systems and methods described herein to the use of a play object to obtain data indicative of player performance.

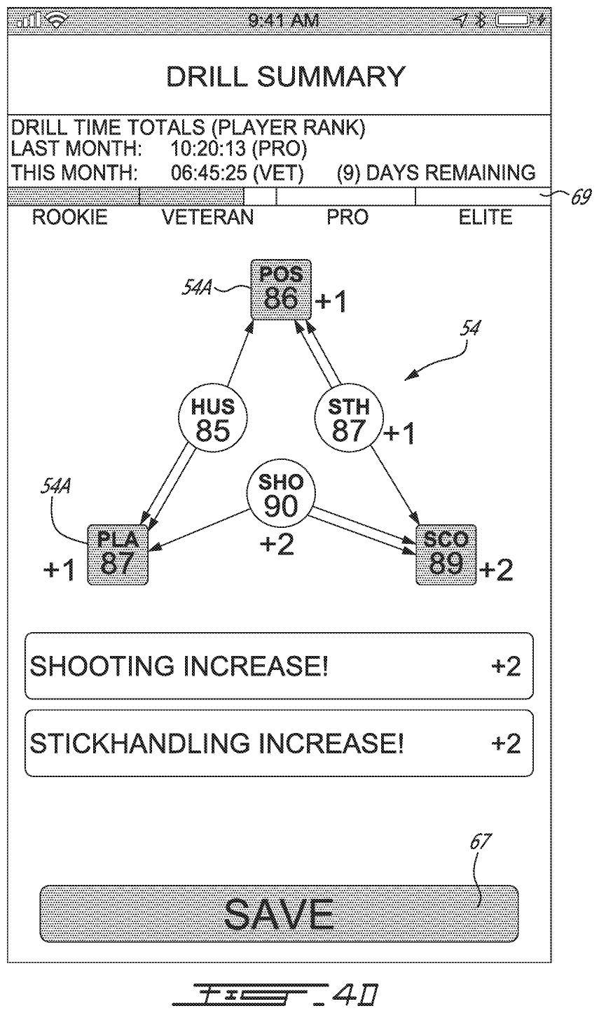

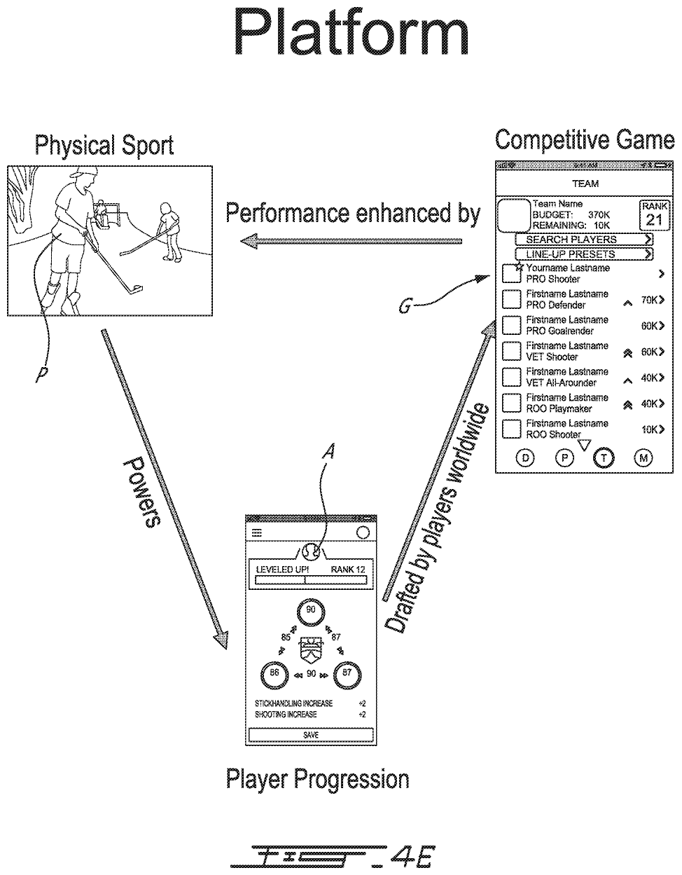

FIG. 3 shows a schematic representation of the computing device 30. In the depicted embodiment, the computing device 30 is a mobile computing device 30A that can be held and transported by the player P. The mobile computing device 30A has a graphical interface that displays the virtual game G and the avatar A of the player P. Examples of the mobile computing device 30A include a mobile telephone, a laptop, and a pad device. The player P can therefore perform the play activity (e.g., manipulate the play object 20) to generate the data indicative of player performance, easily grab the mobile computing device 30A, and interact with the mobile computing device 30A to view and access the virtual game G and avatar A that has been updated by the data indicative of player performance. In an alternate embodiment, the computing device 30 is installed to be stationary, such as a desktop computer.

In one embodiment, the computing device 30 has a display unit, such as a touch-sensitive display 32, which is engaged by the player P. The touch-sensitive display 32 receives inputs I from the player P via tactile interactions therewith, and generates outputs in the form of visual displays of images, icons, charts, alphanumerical characters, and other graphical representations. In the depicted embodiment, the touch-sensitive display 32 is a touchscreen. The computing device 30 has at least one processing unit 34 which is coupled to the touch-sensitive display 32 to receive the inputs I therefrom, and to provide the outputs.

The computing device 30 has a non-transitory computer-readable memory 36 with stored instructions 38 that are executable by the processing unit 34. The non-transitory computer-readable memory 36 may comprise any suitable machine-readable storage medium. The non-transitory computer-readable memory 36 (sometimes referred to herein simply as “memory 36”) may comprise a non-transitory computer readable storage medium, for example, but not limited to, an electronic, magnetic, optical, electromagnetic, infrared, or semiconductor system, apparatus, or device, or any suitable combination of the foregoing. The memory 36 may include a suitable combination of any type of computer memory that is located either internally or externally to the computing device 30, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. The memory 36 may comprise any storage means (e.g., devices) suitable for retrievably storing the machine-readable instructions 38 executable by processing unit 34. The instructions 38 may be in many forms, including program modules, executed by one or more computers or other devices. Generally, program modules include routines, programs, objects, components, data structures, etc., that perform particular tasks or implement particular abstract data types. Typically, the functionality of the program modules may be combined or distributed as desired in various embodiments.

The instructions 38 are executable by the processing unit 34 to communicate at a regular or irregular interval, over a data connection 15 (see FIG. 1), with the data-collecting unit 22 of the play object 20 (and/or with the data collectors 40, 42) to receive therefrom the data indicative of player performance, in either raw and unprocessed, or processed, form. The computing device 30 therefore has a transmitting unit 35 coupled to the processing unit 34, such as an antenna or transceiver, to communicate with the transmitting unit 25 of the data-collecting unit 22. In some embodiments, the transmitting unit 35 is a Bluetooth™ transmitter. The data connection 15 is a server or other network, such as the Internet, a cellular network, Wi-Fi, or others. Furthermore, the transmitting unit 35 may have a signal concentrator (not shown) in communication with the play object 20 and/or the data collectors 40, 42. The signal concentrator may aggregate or concentrate the raw and unprocessed acceleration and rotation values emitted by the play object 20 and/or the data collectors 40, 42, and then relay this concentrated signal data to the processor unit 34 to generate data indicative of player performance. Any known communication protocols that enable devices within a computer network to exchange information may be used. Examples of protocols are as follows: IP (Internet Protocol), UDP (User Datagram Protocol), TCP (Transmission Control Protocol), DHCP (Dynamic Host Configuration Protocol), HTTP (Hypertext Transfer Protocol), FTP (File Transfer Protocol), Telnet (Telnet Remote Protocol), SSH (Secure Shell Remote Protocol).

It will be appreciated that the processing unit 34 may comprise any suitable devices configured to implement the instructions 38 to cause the functionality of the computing device 30 described herein to be implemented. The processing unit 34 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof. The memory 36, processing unit 34, and the instructions 38 are shown as being housed within a physical casing of the computing device 30. In alternate embodiments, the memory 36, the processing unit 34, and the instructions 38 are housed in another type of physical computing device (e.g. tablet, mobile communication device, laptop, etc.). In other alternate embodiments, the memory 36, the processing unit 34, and the instructions 38 are not housed in a physical computing device, and are instead virtually present, such as in a cloud computing system.

Still referring to FIG. 3, the processing unit 34 executes the functions of the computing device 30, and more particularly, of the instructions 38 stored in the memory 36. The processing unit 34 is in communication with each play object 20 and/or with each data collector 40, 42, via a suitable transmitting unit 35 or system transceiver. The processing unit 34 therefore receives from each play object 20 and/or from each data collector 40, 42 the acceleration and rotation values. The processing unit 34 may also emit instructions to one or more play objects 20 and/or data collectors 40, 42. For example, the processing unit 34 can command multiple play objects 20 and/or data collectors 40, 42 to produce movement data along one or more of the X, Y, and Z axes. The processing unit 34 may send signals to deactivate one or more play objects 20 and/or data collectors 40, 42. The processing unit 34 may also configure each play object 20 and/or data collector 40, 42, so as to change for example the sampling frequency at which the acceleration values are measured, to name only a few inputs. The processing unit 34 communicates directly or indirectly with the play objects 20 and/or data collectors 40, 42.

It should be understood that while the instructions 38 presented herein are illustrated and described as separate entities, they may be combined or separated in a variety of ways. The acceleration and rotation values emitted from each play object 20 and/or data collector 40, 42 are received by the computing device 30. The instructions 38 then analyse one or more of these values, along one or more of the degrees of freedom, so as to generate data indicative of player performance. The data indicative of player performance helps to assess a player's skills and abilities or use of the play object 20.

The touch-sensitive display 32 is in communication with the processing unit 34 to visually display the data indicative of player performance, for example as a graphical representation. It will be appreciated that the data indicative of player performance can be provided in a non-graphical format, such as lists of data, a table, etc. Some examples of how the data indicative of player performance is displayed are the following. The touch-sensitive display 32 can display graphs with clear indicators of how the player's performance compares to those of her peers, or that provide a history of player performance over time to track changes in the player's skills. The touch-sensitive display 32 can provide lists of personal “bests” and records. The touch-sensitive display 32 is interactive, and provides a list of player skills that the player P wishes to assess. Some of this include, but are not limited to, “stick handling”, “shot speed”, “best shot”, and “average shot”, “most push-ups in a row”, “fastest time to 10 push-ups”, etc.

Some examples of data indicative of player performance are now described in reference to FIG. 3.

In the embodiment where the play object 20 is a hockey puck 21A, it may be desirable to assess player performance related to how fast the player P can make the hockey puck 21A travel. In such a situation, the instructions 38 executable by the processing unit 34 cause the acceleration values received from the data-collecting unit 22 of the play object 20 along only the X, Y, and Z axes to be processed and an integral thereof to be determined to obtain the associated speed values. The magnitude of these speed values correspond to the maximum speed at which the hockey puck 21A travelled. The computing device 30 in this embodiment therefore generates data indicative of a puck speed when shot by the player P. The instructions 38 are executed by the processing unit 34 to update the avatar A with this data indicative of player performance, such that the avatar A is characterised in the virtual game G with the data indicative of a puck speed when shot by the player P.

In the embodiment where the play object 20 is a hockey puck 21A, it may be desirable to assess player performance related to “quick release”, or how well the player P release a puck without winding-up. In such a situation, the instructions 38 executable by the processing unit 34 process the acceleration values received from the data-collecting unit 22 of the play object 20 along only the X and Y axes using only a high “g” type accelerometer unit, and determines the magnitude of the acceleration values in these axes. Higher acceleration values provide information about the player's “quick release” abilities, for various types of shots (e.g. wrist shot, slap shot, etc.). The acceleration values are combined with the mass of the puck to obtain a force of release of the hockey puck 21A. The computing device 30 in this embodiment therefore generates data indicative of how much force the player P can apply to release a puck. The instructions 38 are executed by the processing unit 34 to update the avatar A with this data indicative of player performance, such that the avatar A is characterised in the virtual game G with the data indicative of how much force the player P can apply to release a puck.

In the embodiment where the play object 20 is a hockey puck 21A, it may be desirable to assess player performance related to “puck stability”, or the ability of the player P to shoot a puck straight. In such a situation, the instructions 38 executable by the processing unit 34 determine the magnitude of the rotation values received from the data-collecting unit 22 of the play object 20 along only the Z axis, and thereby obtain the RPM of the hockey puck 21A about this axis. The RPM is a proxy for puck stability because of the gyroscopic effect, which indicates that the travel path of a fast rotating object will be less perturbed than that of a more slowly rotating object. The computing device 30 in this embodiment therefore generates data indicative of how stably the player P can shoot the hockey puck 21A. The instructions 38 are executed by the processing unit 34 to update the avatar A with this data indicative of player performance, such that the avatar A is characterised in the virtual game G with the data indicative of how stably the player P can shoot the hockey puck 21A.

In the embodiment where the play object 20 is a hockey puck 21A, it may be desirable to assess player performance related to “saucer” passing, which are passes that should be lifted above an opponent while rotating in a substantially horizontal plane so that the hockey puck 21A lands flat on the ice ready for a teammate to shoot or handle. In such a situation, the instructions 38 executable by the processing unit 34 determine the magnitude of the rotation values received from the data-collecting unit 22 of the play object 20 along only the X, Y, and Z axis, and thereby obtain the RPM of the hockey puck 21A about these axes. If the RPM values about the X or Y axes are below a threshold value, then they are indicative of the hockey puck 21A mostly rotating in the Z axis, and thus, in a substantially horizontal plane. The computing device 30 in this embodiment therefore generates data indicative of how well the player P can effectuate a saucer pass. The instructions 38 are executed by the processing unit 34 to update the avatar A with this data indicative of player performance, such that the avatar A is characterised in the virtual game G with the data indicative of how well the player P can effectuate a saucer pass.

In the embodiment where the play object 20 is a hockey puck 21A, it may be desirable to assess player performance related to “stick handling”, which is the ability of the player P to control the hockey puck 21A on the ice. In such a situation, the instructions 38 executable by the processing unit 34 determine the magnitude of the acceleration values received from the data-collecting unit 22 of the play object 20 along only the X and Y axes using a low “g” type accelerometer unit. Low acceleration values coupled with frequent displacements of the hockey puck 21A within the X-Y plane are indicative of the player P having “soft hands”, or good stick handling ability. In contrast, higher acceleration values coupled with fewer displacements of the hockey puck 21A within the X-Y plane can be indicative of the player P having “hands of stone”, or poor stick handling ability. The computing device 30 in this embodiment therefore generates data indicative of how well the player P is able to control the hockey puck 21A. The instructions 38 are executed by the processing unit 34 to update the avatar A with this data indicative of player performance, such that the avatar A is characterised in the virtual game G with the data indicative of how well the player P is able to control the hockey puck 21A.

In the embodiment where the play object 20 is a baseball, it may be desirable to assess player performance related to the “hang” of a pitcher's slider, which is the ability of a ball to move laterally after release without too much vertical movement. In such a situation, the instructions 38 executable by the processing unit 34 manipulate the acceleration values received from the data-collecting unit 22 of the play object 20 of all three of the X, Y, and Z axes using a low “g” type accelerometer unit, and determine an integral of these acceleration values to obtain the velocities along each axis. The instructions 38 executable by the processing unit 34 then determine an integral of these velocities to obtain the displacement of the ball within the X-Y plane, and in the Z axis. If the displacement of the baseball in the X-Y plane exceeds that in the Z-axis (a suitable ratio can be used), then this movement is indicative of a good slider that does not “hang”. The computing device 30 in this embodiment therefore generate data indicative of how well a baseball pitcher is able to throw an effective slider, or any other “spinning” pitch. The system 10 can also track the displacement of a pitch in the X-Y plane and/or the Z axis. This generates data indicative of how well a baseball pitcher is able to throw a pitch (e.g. curveball, sinker, split-finger, change-up) that “drops”, or displaces, in any one of the axes. The instructions 38 are executed by the processing unit 34 to update the avatar A with this data indicative of player performance, such that the avatar A is characterised in the virtual game G with the data indicative of how well a baseball pitcher is able to throw an effective slider, or any other “spinning” pitch.

In the embodiment where the play object 20 is a hockey puck 21A, it may be desirable to assess player performance related to how often the player P touches the hockey puck 21A. In such a situation, the instructions 38 executable by the processing unit 34 use frequent displacements of the hockey puck 21A within the X-Y plane as data indicative of the frequency at which the player P touches or handles the hockey puck 21A. The computing device 30 in this embodiment therefore generates data indicative of how often the player P touches the hockey puck 21A. The instructions 38 are executed by the processing unit 34 to update the avatar A with this data indicative of player performance, such that the avatar A is characterised in the virtual game G with the data indicative of how often the player P touches the hockey puck 21A. This data is “usage” data, in that it is not a measure of the skill of the player P but simply a measure of how often the player P is using the hockey puck 21A.

It will be appreciated that the embodiments described above of data indicative of player performance are not limiting, and that other types of such data, from hockey as well as from other sports, are within the scope of the present disclosure. In particular, and as discussed herein above, the systems and methods described herein are not limited to the use of a play object as in 20 for obtaining data indicative of player performance. In some embodiments, no play object 20 is used by the player P during the play activity and player performance is assessed based on data obtained from data collectors as in 40, 42 provided on the player's body or in a personal electronic device of the player P.

Still referring to FIG. 3, the instructions 38 stored in the memory 36 of the computing device 30 are also executable by the processing unit 34 to store the data indicative of player performance in the memory 36, and to update the avatar A in the virtual game G based on the data indicative of player performance stored in the memory 36 and/or received (e.g., from the data-collecting unit 22 of the play object 20 or from the data collectors 40, 42). The memory 36 also stores the virtual game G and the avatar A of the player P. The virtual game G is a digital environment or space. The virtual game G and avatar A exist only in the memory 36 of the computing device 30, and the instructions 38 executable by the processing unit 34 modify the virtual game G and the avatar A based on the data indicative of player performance as received.

The term “update” or “modify” refers to the instructions 38 executing on the processing unit 34 to change one or more characteristics or attributes associated with the avatar A of the player P in the virtual game G, based on the real-world data generated by the player P (e.g., while manipulating the play object 20). The characteristics may include, but are not limited to, at least one of a profile, a skill level, a duration of play time, etc. of the avatar A. The updates or revisions to the avatar A are stored in the memory 36. It can therefore be appreciated that the play object 20 and/or the data collectors 40, 42 generate data on the performance of the player P, feed this data indicative of player performance into the virtual game G, and the computing device 30 uses data indicative of player performance to modify the avatar A. In the depicted embodiment, the avatar A and its activities within the virtual game G are not controlled or dictated by the player P manipulating the play object 20. Instead, the performance of the player P of play activities using the play object 20, or mere use of the player object 20 by the player P, helps to “build up” or improve the avatar A, which can then subsequently and separately be used in the virtual game G. In yet another embodiment where no play object 20 is used during the play activity and the data indicative of player performance is instead received from the player's personal electronic device (e.g., from the data collectors 40, 42 provided therein), the personal electronic device is then used as the equivalent of a virtual game controller to similarly improve the avatar A. In this manner, the system 10 transforms the physical activity of the player P into something beneficial for the representation of the same player P in the virtual game G.

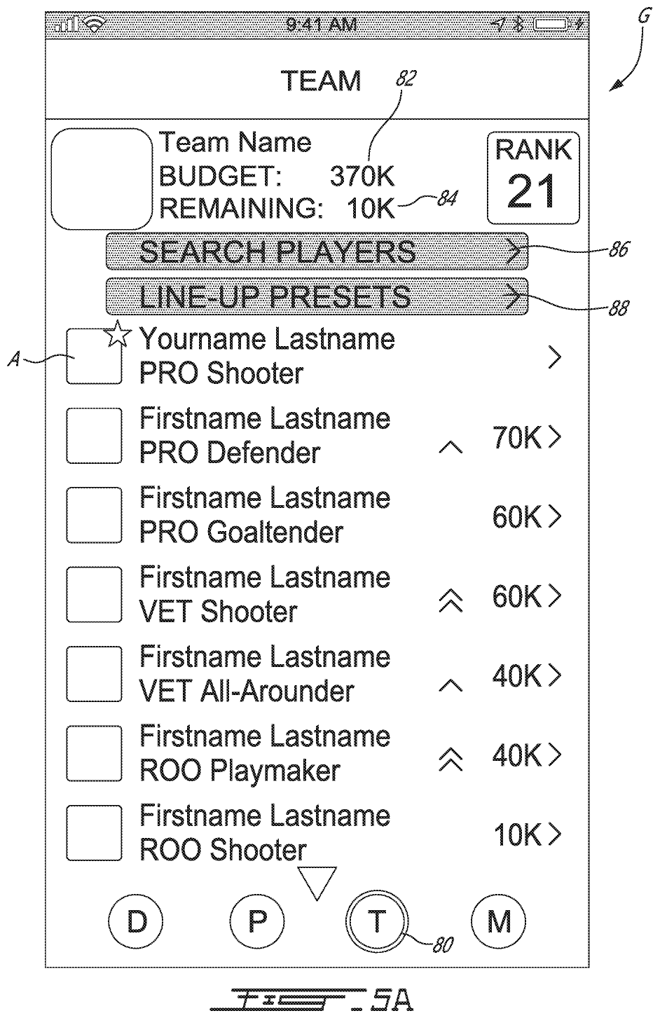

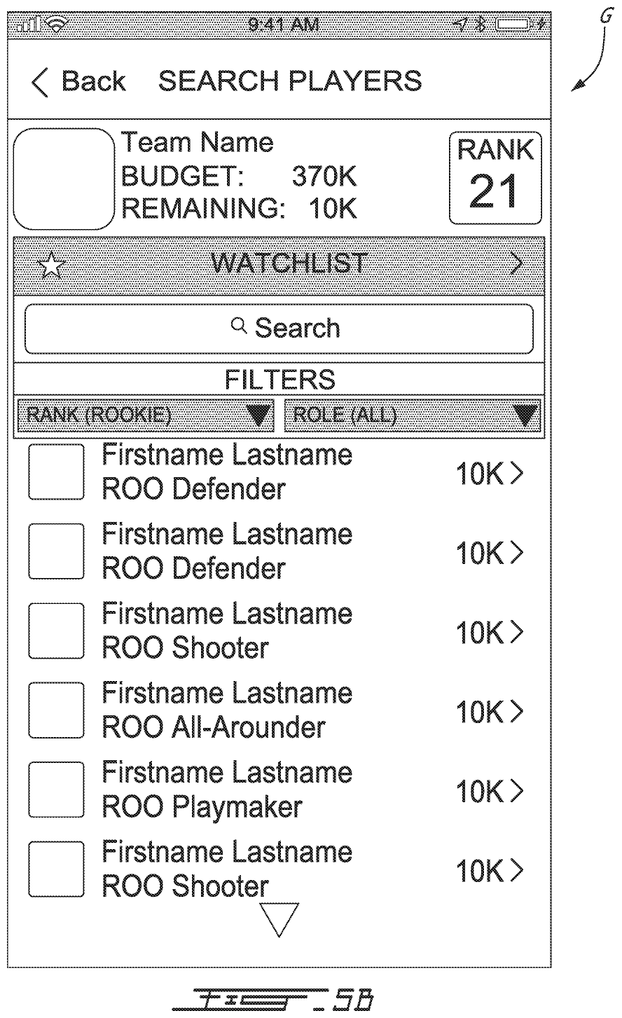

Examples of the virtual game G, the avatar A, and the updating thereof with data indicative of player performance are now described in greater detail with reference to FIGS. 4A to 6C.

FIG. 4A shows a landing page of the virtual game G, as displayed on the touch-sensitive display 32 of the mobile computing device 30A. The virtual game G is related to the sport of hockey. The landing page displays a profile of the avatar A, which in the depicted embodiment, is a hockey player. The profile shows the skill level 50 of the avatar A, and the type of player 52 that the avatar A is (e.g. a shooter). The profile also shows a skills matrix 54, which is an amalgamation of the skills of the avatar A that are relevant to the virtual game G. The skills matrix 54 includes six skills: three player skills 54A, and three drill skills 54B.

The drill skills 54B are used to determine the player skills 54A. In the embodiment of FIG. 4A, the drill skills 54B of the virtual game G are “SHO” for shooting, “STH” for stick handling, and “HUS” for hustle. In one embodiment, the drill skills 54B are determined through assessment of data (e.g., acceleration and rotation values) obtained from the play object 20 in the manner described above with reference to FIGS. 1 to 3). In another embodiment, the drill skills 54B, and more particularly STH, are determined through assessment of the data obtained from (e.g., from data collectors as in 40, 42 provided in) the personal electronic device of the player P.

One of the player skills 54A in the depicted embodiment is “POS”, which is an indicator of the ability of the avatar A to gain and maintain possession of the puck in the virtual game G. As can be seen, the drill skills 54B of HUS and STH determine the value for the player skill 54A POS, and the drill skill 54B STH is weighted more than the drill skill 54B STH as indicated by the two arrows for STH versus the single arrow for HUS. Another one of the player skills 54A is “PLA”, which represents playmaking and the ability of the avatar A to create scoring chances whenever its team has possession of the puck in the virtual game G. The drill skills 54B of HUS and SHO factor into determining the value for the player skill 54A of PLA.

Still referring to FIG. 4A, the profile of the avatar A also shows its statistics 56 over the course of its career in the virtual game G. When the player P engages the touch-sensitive display 32 to click on the shift summaries icon 58, the touch-sensitive display 32 will respond to this input to display a page of the virtual game G which shows how the avatar A participated in recent matches it played in the virtual game G. When the player P engages the touch-sensitive display 32 to click on the teams icon 59, the touch-sensitive display 32 will respond to this input from the player P to display a page of the virtual game G which shows teams that the avatar A is part of.

The following icons are displayed along the bottom of the profile page: a drills icon 60, a player profile icon 70 (engagement of which prompts the touch-sensitive display 32 to display the profile page shown in FIG. 4A), a team icon 80, and a matches icon 90. These graphical user interfaces will now be described in greater detail.

Referring to FIGS. 4B to 4D, when the player P engages the touch-sensitive display 32 to click on the drills icon 60, the touch-sensitive display 32 will respond to this input to display a page of the virtual game G which shows drills that the player P has performed during the play activities, and which have been stored in the memory 36 of the computing device 30 (see FIG. 4B). Each drill is a play activity performed by the player P with or without the play object 20 in the real, non-virtual environment. For instance, some drills may be shooting drills that require the play object 20 while other drills may be fitness drills that do not required the play object 20 but only require the personal electronic device (e.g., the mobile phone) of the player P. The page of the drills icon 60 shows recent drills and their duration. To have a new drill recorded in the virtual game G, the player P engages the touch-sensitive display 32 to click on the start icon 62. The touch-sensitive display 32 will respond to this input by displaying a timer which will record the duration of the drill being performed by the player P until the player P engages the touch-sensitive display 32 to click on the start or stop icon 62 again. In an alternate embodiment, the player P does not actively engage the computing device 30 to start the drill. The computing device 30 in this embodiment is alerted to start of a drill when the play object 20 is manipulated by the player P.

When the player P has finished the play activity and engages the touch-sensitive display 32 to stop recording the drill by clicking on the start or stop icon 62, the touch-sensitive display 32 will respond to this input by displaying a drill summary page of the virtual game G (see FIG. 4C). The drill summary page shows a breakdown of the drill performed by the player P into different indicators of player performance 64 (e.g. number of shots, release time of shots, shot speed, dribble streak, etc.), in the case of a drill requiring the use of a play object 20. Each of these indicators of player performance 64 is given a value or grade, and assigned to one of the drill skills 54B described above. The drill summary page also displays a grade 66 assigned to the avatar A for the drill. The grade 66 is determined based on the data indicative of player performance generated by the data-collecting unit 22 of the play object 20. The average of the grades 66 earned for all drills helps to determine a rank of the avatar A in the virtual game G.

When the player P engages the touch-sensitive display 32 to click the next icon 68, the touch-sensitive display 32 will respond to this input by displaying a page explaining how recently-completed drill factors into the profile of the avatar A (see FIG. 4D). A progression bar 69 shows the rank of the avatar A over a given period (e.g. a month) based on the duration of the drills performed by the player P over the same period. The rank of the avatar A is directly related to the duration of play activities performed by the player P over the time period. Other characteristics of the play activity by the player P may also influence the rank of the avatar A in the virtual game G (e.g., such as performance or skill of the player P when performing the play activities). For example, in the depicted embodiment of the virtual game G, if the player P performs over ten hours of play activities on the play object 20 in the time period, the avatar A will achieve the rank of “PRO”. In the skills matrix 54 shown in FIG. 4D, the value of the POS and PLA player skills 54A has increased because of the play activity recently completed by the player P. When the player P engages the touch-sensitive display 32 to click the save icon 67, this input will cause the processing unit 34 to execute the instructions 38 to save the latest drill to the memory 36. This input will also cause the touch-sensitive display 32 to display the profile page of FIG. 4A with updates to reflect new values for the player skills 54A.

It can therefore be appreciated that the performance of the play activity by the player P will cause the instructions 38 executable by the processing unit 34 to update the avatar A of the player P by increasing or decreasing a rank of the avatar A in the virtual game G. The performance of the play activity by the player P will also cause the instructions 38 executable by the processing unit 34 to update the avatar A of the player P by increasing or decreasing a skill level 54A, 54B of the avatar A in the virtual game G. These updates to the avatar A are based on a duration of time that the player P spends manipulating the play object 20. It can therefore be appreciated that for the virtual game G in the depicted embodiment, it is the amount of real-world physical activity by the player P, specifically the time spent performing play activities, that determines the profile, skills, and features of the avatar A in the virtual game G. This technique for improving the avatar A provides an incentive for the player P to perform real-world exercise and activities because the more the player P performs the play activities, the better the avatar A will become. As shown in FIG. 4E, the physical activity of the player P in the real, non-virtual environment powers the avatar A in the virtual game G. The virtual game G also communicates with the player P in the real, non-virtual environment to encourage the player P to perform more or improved play activities. The performance of the player P in the real, non-virtual environment is therefore enhanced by the virtual game G.

Between drills, the player P may also play the virtual game G using her avatar A, and other avatars as well. The depicted embodiment of the virtual game G allows the player P to take on the role of captain or manager of a team. The teams in the virtual game G consist of other avatars A which are not representations of the player P playing the game, but of other players P using other play objects 20. The player P is therefore able to play the virtual game G to captain or manage a team of avatars A. In the depicted embodiment of the virtual game G, the avatar A of the player P is on many teams, but the player P may only manage one team. In an alternate embodiment, the player P manages more than one team in the virtual game G.

As another kind of embodiment of the interaction between the real and digital worlds, the physical activity performed by a player P could be used as currency in any virtual game that has a currency. For example, the virtual game could be a city simulator type of game (e.g., SimCity™) that could incorporate a so-called “sweat resource”. In this case, the usual kind of resources would be required from the player P but some sweat resource would also be required to build out a particular piece of the city. The systems and methods disclosed herein could also be used in any role playing game where the attributes of a given character (e.g., the player's avatar A) are related to the in-game progress. For example, advancing in quests or killing monsters would provide an experience that the player P may put into the strength attribute of their avatar A. In other words, instead of making the player's experience dependent only on in-game virtual accomplishments, real world completion of tasks may be required to level up the strength of the player's character. For many sporting games, the player's performance in the real world could enhance the attributes of the avatar A in the digital world, as discussed herein above. Furthermore, in each of these virtual game types, a multiplayer element (be it for trading, competition or cooperation) may be included that could involve not only the play in the virtual world, but also exchanging real world effort components (e.g., trading push-ups for squats to unlock a specific item) or competing in a virtual battle where a simultaneous completion of real world activity gives a significant boost to characters. Another example is a virtual trivia game where players P typically get online at the same time to answer questions. In the virtual trivia game, physical activity would instead be used to answer the questions.

To offer another kind of embodiment of the interaction between the real world and the digital one, it would also be possible to have the real world efforts of the player P allow unlocking of various pieces of content in the virtual game G. Many mobile games require players to build objects in the game while utilizing resources to build those objects. One of those resources could be the real world physical effort (i.e., the user-generated motion performed during the play activity) done by a user in the real world. One example would be the player P having to perform a given number of (e.g., ten (10)) push-ups in the non-virtual environment in order to build a virtual object (e.g., a castle) in the virtual environment of the virtual game G. Physical effort could also reduce the cost or the cooldown of activities that usually constitute in-app purchases for mobile games. For example, if the player P is out of lives in the virtual game G, the player P may be required to either (1) wait for a predetermined time period (e.g., one hour) to elapse before being allowed to resume play or (2) save two minutes per push-up completed in the non-virtual environment. Other embodiments may apply.

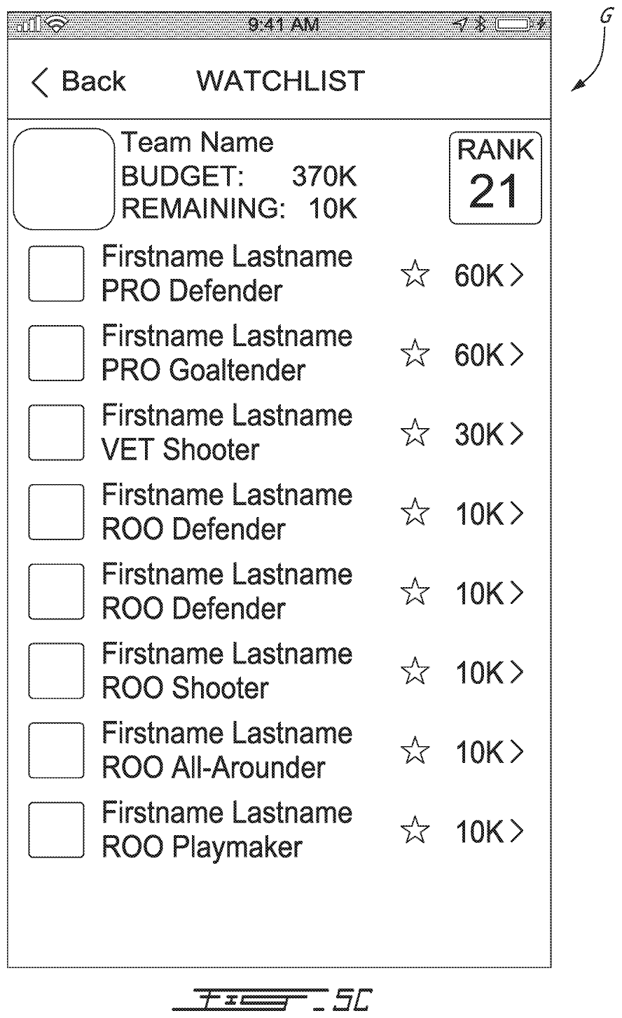

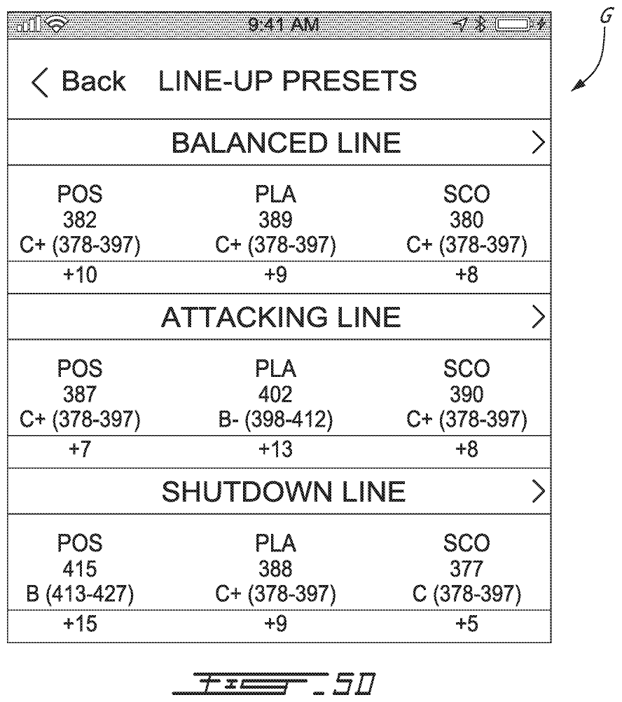

Referring to FIGS. 5A to 5D, when the player P engages the touch-sensitive display 32 to click the team icon 80, the touch-sensitive display 32 will respond to this input by displaying a page showing the composition of a team in the virtual game G being managed by the player P (see FIG. 5A). The page shows the roster of avatars A that make up the team. The page also shows the budget 82 of the team which indicates how much the player P has “spent” to form the team, and the remaining budget 84 which the player P can use to acquire other avatars. When the player P engages the touch-sensitive display 32 to click the search players icon 86, the touch-sensitive display 32 will respond to this input by displaying a page showing the other avatars in the virtual game G that can be added to roster of the player's team (see FIG. 5B). When the player P engages the touch-sensitive display 32 to click the name of another avatar, the touch-sensitive display 32 will respond to this input by displaying the “cost” of acquiring the other avatar for the team of the player P (see FIG. 5C). In the virtual game G of the depicted embodiment, a “Shooter” is an avatar who is good at scoring, a “Defender” is one who is good at stick handling, a “Goaltender” is a goalie, a “Playmaker” is one who is good at moving puck to the opponent's zone, and an “All-Rounder” is one who has no specialty but who is good at filling in gaps.

When the player P engages the touch-sensitive display 32 to click the line-up presents icon 88, the touch-sensitive display 32 will respond to this input by displaying the other avatars making up a line-up for a match in the virtual game G (see FIG. 5D). Similar to the real sport of hockey, in the virtual game G it is possible to have different lines made up of different avatars for strategic purposes. Referring to FIG. 5A, each season in the virtual game G of the depicted embodiment lasts about three months, for example, at which point the team of the player P will be assigned a rank. The rank is determined as a function of the number of times matches are played, and the number of winning outcomes of each match to incentivize both playing the virtual game G a lot and playing well. As the rank of the team increases, so does its budget, and the player P can therefore select more skilled avatars for her team.

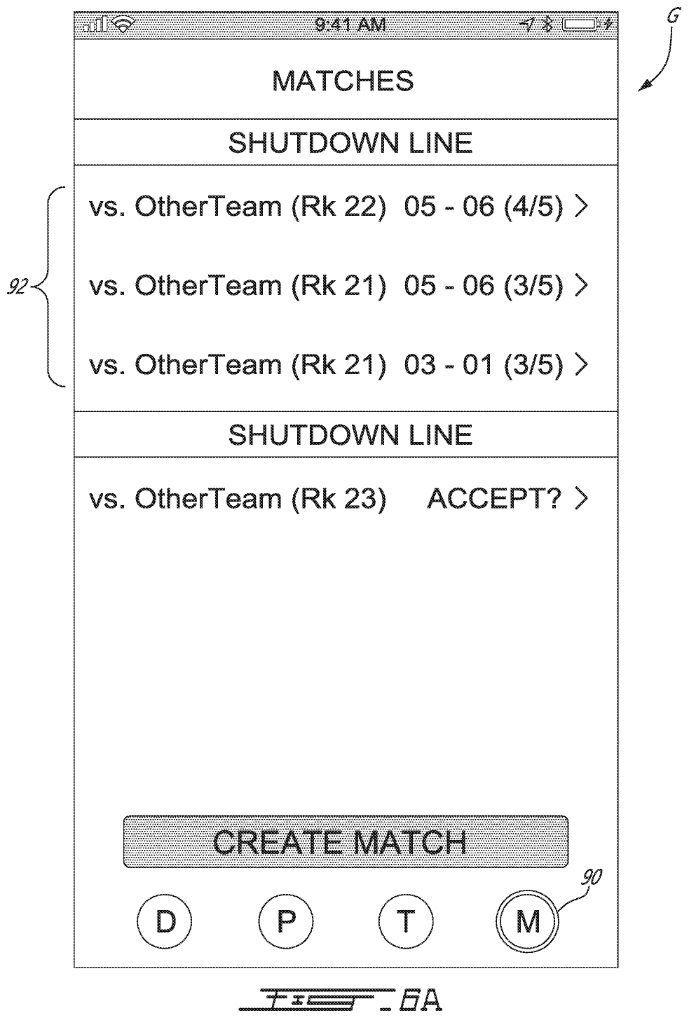

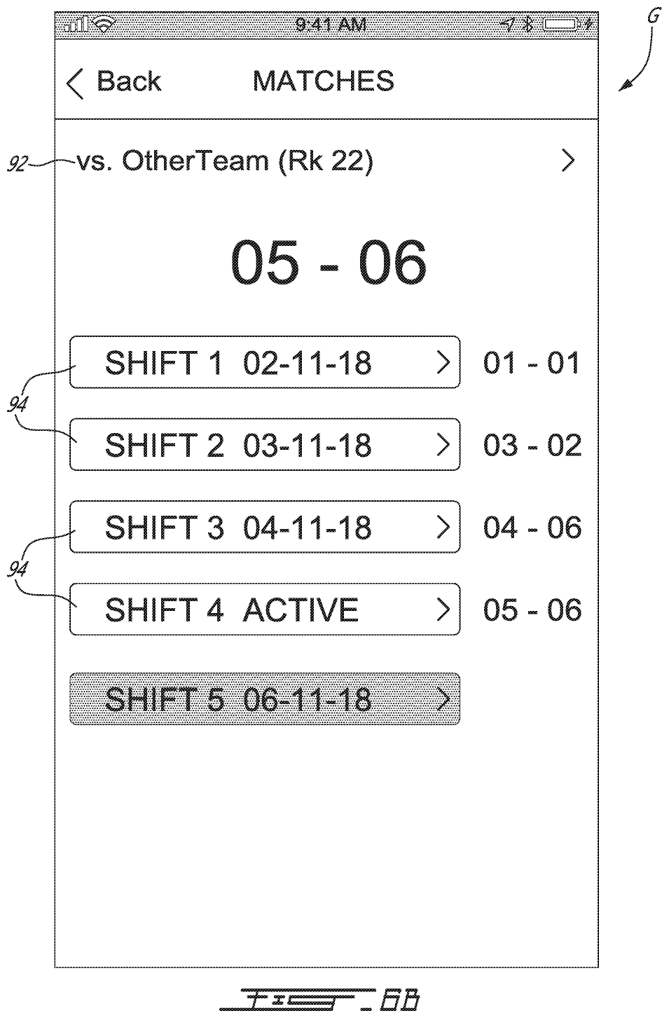

Referring to FIGS. 6A to 6C, when the player P engages the touch-sensitive display 32 to click the matches icon 90, the touch-sensitive display 32 will respond to this input by displaying a page showing the current matches 92 that the team of the player P is playing in the virtual game G, as well as matches 92 to which the team of the player P has been invited (see FIG. 6A). The player P can also create a match 92 between teams. When the player P engages the touch-sensitive display 32 to click on one of the matches 92, the touch-sensitive display 32 will respond to this input by displaying a page that shows the match 92 (see FIG. 6B). Each match 92 is broken down into a series of shifts 94. FIG. 6B shows the score at the end of each shift 94, and which shift 94 is current or active. In the virtual game G of the depicted embodiment, one shift 94 is played per day. When the player P engages the touch-sensitive display 32 to click on one of the shifts 94, the touch-sensitive display 32 will respond to this input by displaying a page which breaks down the activities that occurred during that shift 94 (see FIG. 6C). This shift summary page breaks down each shift 94 into the possessions 96 that occurred during the shift 94. In the depicted embodiment, a shift 94 is made up of six or seven possessions 96. The player P can watch a shift 94 play out in real time by checking the shift summary page every few minutes. FIG. 6C shows one implementation of the virtual game G where the player P may follow the virtual game G in a text-based format that would provide notifications of recent actions.

When the player P engages the touch-sensitive display 32 to click on the line-up icon 98, the touch-sensitive display 32 will respond to this input by displaying the other avatars which make up the line-up for that shift 94. The player P can change the line-up of avatars, and the virtual game G may incentivize the player P to manage the different line-ups well by penalizing the team if the same avatars are present on all shifts 94. In this manner, the player P is incentivized to strategize as a captain or manager would in a real hockey game. It can therefore be appreciated that real-word physical exercise with the play object 20 allows the avatar A to acquire skills in the virtual game G that will help its profile and the performance of the teams it plays on. The virtual game G may actively reinforce this feedback mechanism by providing advantages to players P who practice before or during the virtual game G. Additional advantages may be given to teammate avatars who practice at the same time creating a collective pressure to get many people together to play collectively.

Other embodiments of virtual game G are possible. Reference herein to hockey pucks or skills associated with hockey in the virtual game G does not limit the disclosed virtual game G to being only related to hockey. Other implementations of the virtual game G link other real-world activities to virtual environments. The play object 20 can be used to generate mana or hit points in a role-playing game (RPG) or to generate money in a strategy time game.

In an embodiment, the system 10 includes an audio output device that is wearable by the player P. The instructions 38 are executable by the processing unit 34 to communicate with the audio output device and provide audio status information about the updates to the avatar A in the virtual game G. This earpiece or headphone would provide live (i.e., substantially real-time) progress tracking and encouragement that would stem from the analysis of the data coming from the play object 20. The audio output device provides the player P with audible signals representative of changes to the avatar A. For example, the audio output device may provide the player P with audible signals by which the player P is updated on the progress of the avatar A, such as “only ten more shots to level up your avatar”. The audio output device therefore provides feedback to the player P in the real, non-virtual environment directly from the virtual game G.

In at least some of the embodiments described above, the player P and the virtual game G are not in sync. In an alternate embodiment, the player P and the virtual game G are in sync. In one possible example of this operating mode, the avatar A receives possession of the puck in the virtual game G. The virtual game G signals the player P advising them of this development, for example through the audio output device or the touch-sensitive display 32, and provides the player P with a time limit to take a real shot with the hockey puck 21A. The data from the shot taken by the player P in the real, non-virtual environment will be fed back into the virtual game G to determine what the avatar A will do in the virtual game G. In this embodiment, the virtual game waits for input from the player P via the play object 20.

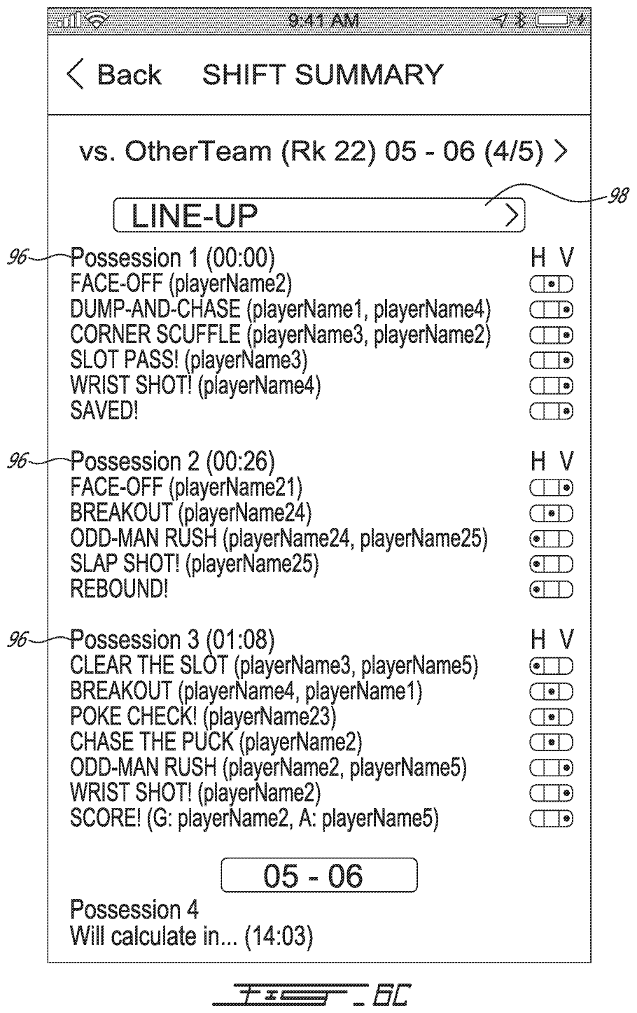

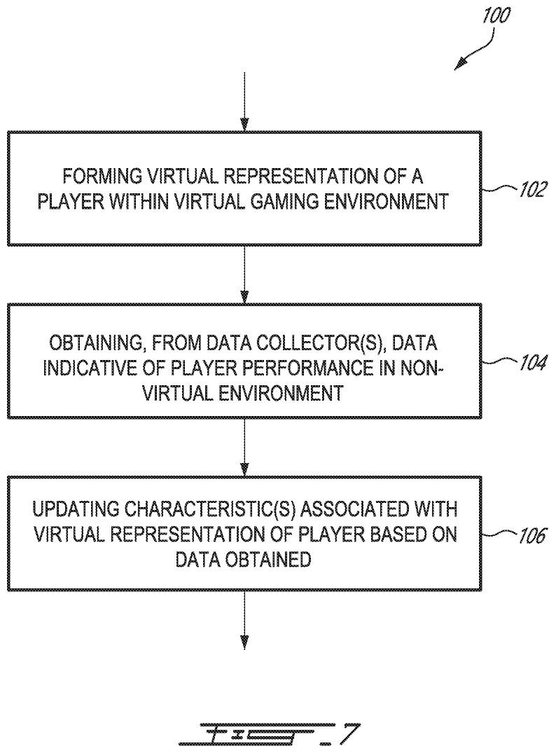

Referring now to FIG. 7, a method 100 for tracking a player performing a play activity in a non-virtual environment and mapping the play activity into a virtual environment will now be described. The method 100 is illustratively implemented on the computing device 30 of FIG. 1. At step 102, a virtual representation (also referred to herein as an avatar) of the player is formed within the virtual environment of a virtual game. One or more characteristics are associated with the virtual representation of the player, as formed. Data indicative of the player's performance in the non-virtual environment is then obtained at step 104 from one or more data collectors, such as one or more play objects (reference 20 in FIG. 1) and/or one or more data collectors (references 40 and 42 in FIG. 1) located on the player's body or provided in the player's personal electronic device (e.g., mobile phone). The data may then be used at step 106 to update the characteristic(s) associated with the virtual representation of the player. As discussed above, the data obtained at step 104 may comprise information related to the skill or abilities of the player (e.g., when manipulating the play object), and/or raw or unprocessed data (e.g., acceleration and/or rotation values indicative of a movement of the play object) that can be processed into information related to the skill or abilities of the player (e.g., when manipulating the play object). As also discussed above, step 106 illustratively comprises using the obtained data to “build up” or improve the virtual representation of the player, which can then subsequently be used in the virtual game.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure, and such modifications are intended to fall within the appended claims.

1. A system for mapping a play activity performed by a player in a non-virtual environment into a virtual gaming environment, the system comprising:
at least one data collector configured to collect, at least during the play activity, data indicative of a performance of the player; and
a computing device comprising:
at least one processing unit, and
at least one non-transitory computer-readable memory having stored thereon a virtual game and a virtual representation of the player within a virtual environment of the virtual game, the virtual representation of the player having one or more characteristics associated therewith, the memory also having stored thereon instructions executable by the at least one processing unit for:
obtaining, from the at least one data collector, the data indicative of the performance of the player, and
updating, based on the data indicative of the performance of the player, the one or more characteristics associated with the virtual representation of the player.

2. The system of claim 1, wherein the at least one data collector is at least one play object manipulable by the player during the play activity.

3. The system of claim 2, wherein the at least one play object is a connected sports object.

4. The system of claim 1, wherein the at least one data collector is at least one motion sensing device located on a body of the player.

5. The system of claim 1, wherein the at least one data collector is at least one motion sensing device provided in a portable communication device of the player.

6. The system of claim 2, wherein the at least one play object comprises a data collecting unit configured to collect the data indicative of the performance of the player and to output the data to the computing device.

7. The system of claim 6, wherein the data collecting unit comprises:
at least one accelerometer unit configured for measuring acceleration values of the at least one play object along at least one translational degree of freedom;
at least one gyroscope unit configured for measuring rotation values of the at least one play object about at least one rotational degree of freedom; and
a processor in communication with the at least one accelerometer unit and with the at least one gyroscope unit, the processor configured for obtaining the acceleration values from the at least one accelerometer unit, obtaining the rotation values from the at least one gyroscope unit, and outputting the acceleration values and the rotation values to the computing device at discrete intervals.

8. The system of claim 7, wherein the at least one processing unit is configured for receiving the acceleration values and the rotation values and for determining therefrom the data indicative of the performance of the player.

9. (canceled)

10. (canceled)

11. The system of claim 1, wherein the instructions are executable by the at least one processing unit for updating the one or more characteristics comprising increasing or decreasing a rank associated with the virtual representation of the player in the virtual game.

12. The system of claim 1, wherein the instructions are executable by the at least one processing unit for updating the one or more characteristics comprising increasing or decreasing a skill level associated with the virtual representation of the player in the virtual game.

13. (canceled)

14. The system of claim 1, wherein the instructions are executable by the at least one processing unit for updating the one or more characteristics comprising increasing a duration of play time associated with the virtual representation of the player in the virtual game.

15. The system of claim 1, further comprising an audio output device wearable by the player, the instructions executable by the at least one processing unit for outputting in substantially real-time, via the audio output device, audio status information about any update to the virtual representation of the player.

16. The system of claim 1, wherein the computing device further comprises a display unit configured to render thereon the data indicative of the performance of the player.

17. A computer-implemented method for mapping a play activity performed by a player in a non-virtual environment into a virtual gaming environment, the method comprising, at a computing device:
forming a virtual representation of the player within a virtual environment of a virtual game, the virtual representation of the player having one or more characteristics associated therewith;
obtaining, from at least one data collector, data indicative of a performance of the player; and
updating the one or more characteristics associated with the virtual representation of the player based on the data as obtained.

18. The method of claim 17, wherein obtaining the data indicative of the performance of the player comprises:
obtaining, from at least one accelerometer unit, acceleration values of the at least one data collector along at least one translational degree of freedom;
obtaining, from at least one gyroscope unit, rotation values of the at least one data collector about at least one rotational degree of freedom; and
determining the data indicative of the performance of the player from the acceleration values and the rotation values.

19. The method of claim 17, wherein updating the one or more characteristics comprises increasing or decreasing a rank associated with the virtual representation of the player in the virtual game.

20. The method of claim 17, wherein updating the one or more characteristics comprises increasing or decreasing a skill level associated with the virtual representation of the player in the virtual game.

21. (canceled)

22. The method of claim 17, wherein updating the one or more characteristics comprises increasing a duration of play time associated with the virtual representation of the player in the virtual game.

23. (canceled)

24. (canceled)

25. A computing device comprising:
at least one processing unit; and
at least one non-transitory computer-readable memory having stored thereon a virtual game and a virtual representation of the player within a virtual environment of the virtual game, the virtual representation of the player having one or more characteristics associated therewith, the memory also having stored thereon instructions executable by the at least one processing unit for:
obtaining, from the at least one data collector, data indicative of a performance of a player during a play activity, and
updating the one or more characteristics associated with the virtual representation of the player based on the data as obtained.

26. (canceled)

27. (canceled)

28. (canceled)

29. (canceled)

30. (canceled)

31. (canceled)

32. (canceled)

33. (canceled)

34. (canceled)

35. A non-transitory computer-readable medium having stored thereon a virtual game and a virtual representation of a player within a virtual environment of the virtual game, the virtual representation of the player having one or more characteristics associated therewith, the computer-readable medium also having stored thereon program code executable by at least one processor for:
obtaining, from at least one data collector, data indicative of a performance of a player during a play activity, and
updating the one or more characteristics associated with the virtual representation of the player based on the data as obtained.
